(19) United States
(12) Patent Application Publication
(10) Pub. No.: US 2023/0190225 A1
(43) Pub. Date: Jun. 22, 2023

(54) INTRAVASCULAR IMAGING ASSESSMENT OF STENT DEPLOYMENT AND ASSOCIATED SYSTEMS, DEVICES, AND METHODS

(71) Applicant: PHILIPS IMAGE GUIDED THERAPY CORPORATION, San Diego, CA (US)

(72) Inventors: Asher COHEN, SAN DIEGO, CA (US); Pei-Yin CHAO, EINDHOVEN (NL); Ronald HELMSTRIJD, EINDHOVEN (NL)

(21) Appl. No.: 18/082,303

(22) Filed: Dec. 15, 2022

Related U.S. Application Data
(60) Provisional application No. 63/290,688, filed on Dec. 17, 2021.

(51) Int. Cl. A61B 8/08; A61B 8/12; A61B 8/00; G06V 10/44; G06T 7/60; G06T 11/00

(52) U.S. Cl. CPC A61B 8/0841; A61B 8/12; A61B 8/463; G06V 10/44; G06T 7/60; G06T 11/00; G06V 2201/034; G06T 2207/30101; G06T 2210/41

(57) ABSTRACT

A system includes a processor circuit that receives intraluminal images of a body lumen. The processor circuit identifies a stent edge within the intraluminal images and calculates a distance between the stent edge and the vessel wall. The processor circuit additionally compares the distance between the stent edge and the vessel for the intraluminal images to a threshold distance and identifies which intraluminal images correspond to a distance exceeding the threshold. The processor circuit further outputs to a display a longitudinal view of the body lumen identifying locations along the stent at which the distance between the stent and the vessel wall exceeds the threshold distance.

### CROSS-REFERENCE TO RELATED APPLICATIONS

This application claims priority to and the benefit of U.S. Provisional Application No. 63/290,688, filed Dec. 17, 2021, which is incorporated by reference herein in its entirety.

### TECHNICAL FIELD

The present disclosure relates generally to post-deployment stent assessment. In particular, areas of a stent with a higher risk of post-deployment complications are automatically identified based on a distance between a stent edge and a vessel wall.

### BACKGROUND

Physicians use many different medical systems and tools to monitor a patient's health and diagnose and treat medical conditions. Different modalities of medical diagnostic systems may provide a physician with different images, models, and/or data relating to internal structures within a patient. These modalities include invasive devices and systems, such as intravascular systems, and non-invasive devices and systems, such as x-ray systems, and computed tomography (CT) systems. Treatment procedures also include invasive and non-invasive treatment devices and systems designed to increase or restore blood flow in constricted blood vessels.

In the field of intravascular treatment procedures, stents are commonly used by physicians to restore blood flow. During a stent deployment procedure, a stent may be placed within a vessel so that a central portion of the stent is placed at a region of restricted blood flow. While stents remain an effective and widely adopted tool in restoring blood flow in various vessels of the body, the deployment of a stent also presents a risk of post-deployment complications. Some post-deployment complications include a stent becoming dislodged, deformed, or compressed, among others.

Post-dilatation is a procedure in which a vessel, or a stent within a vessel, may be expanded or re-expanded after it has become compressed. Post-dilatation may be accomplished via many difference medical treatment devices. However, currently, physicians are provided with few tools to assist them in assessing whether a stent is in need of post-dilatation after deployment or if areas of a stent may correspond to a higher risk of future complications.

### SUMMARY

Embodiments of the present disclosure are systems, devices, and methods for assessing stent deployment. Aspects of the present disclosure advantageously assist a physician in assessing whether a stent is correctly positioned and is properly expanded. Aspects of the present disclosure may also assist a physician in identifying stents, or regions of a stent, that are more susceptible to future complications. Features of the present disclosure may also advantageously assist a physician in determining a post-dilatation plan and/or prognostic values. After a stent is deployed in a blood vessel, a physician may perform an intravascular ultrasound (IVUS) imaging procedure. During this procedure, the IVUS imaging device may pass through the stent. The processor circuit then identifies all IVUS images which show a stent and identifies a vessel wall and a stent edge for each IVUS image.

After a vessel wall and stent edge are identified in all applicable IVUS images, the processor circuit identifies which IVUS images show areas of a stent in need of post-dilatation or areas of a stent which pose a higher risk of future complications. In one example, the processor circuit calculates a distance between the vessel wall and the stent edge for one or more locations around the vessel. The processor circuit then compares these distance measurements to a threshold distance. The processor circuit then identifies any images in which one or more distance measurements exceed the threshold. In another example, the processor circuit determines the cross-sectional area of the vessel and the cross-sectional area of the stent or lumen of each IVUS image to determine a plaque burden for each IVUS image. The plaque burden for each IVUS image is then compared to a threshold. The processor circuit may then identify all IVUS images with a plaque burden exceeding the threshold.

The processor circuit then identifies all locations along the stent which exceed either of these thresholds and displays them to a user of the system. These locations identify for a physician areas of the stent which may need to be re-expanded or otherwise treated with a post-dilatation procedure. These locations may also inform a physician of an increased risk of future complications related to the stent.

In an exemplary aspect of the present disclosure, a system is provided. The system includes a processor circuit configured for communication with an intraluminal imaging device, the processor circuit configured to: receive a plurality of intraluminal images obtained by the intraluminal imaging device during the movement of the intraluminal imaging device within a body lumen of a patient; identify a stent edge of a stent and a vessel wall within the plurality of intraluminal images; calculate a distance between the stent edge and the vessel wall for the plurality of intraluminal images; compare, for the plurality of intraluminal images, the distance between the stent edge and the vessel wall with a threshold distance; identify a subset of the plurality of intraluminal images corresponding to the distance between the stent edge and the vessel wall satisfying the threshold distance; output, to a display in communication with the processor circuit, a screen display comprising a longitudinal view of the body lumen, wherein the longitudinal view of the body lumen comprises: a graphical representation of the stent; and locations indicated along the stent corresponding to the subset of intraluminal images.

In some aspects, the processor circuit is further configured to automatically analyze the plurality of intraluminal images to determine whether the stent edge is present within the plurality of intraluminal images. In some aspects, the processor circuit is configured to perform the identification of the stent edge and the vessel wall and the calculation of the distance between the stent edge and the vessel wall in response to automatically determining that the stent edge is present in the plurality of intraluminal images. In some aspects, the processor circuit is further configured to determine a stent area for the plurality of intraluminal images. In some aspects, the processor circuit is further configured to identify a location along the longitudinal view corresponding to a minimum stent area. In some aspects, the processor circuit is further configured to display an intraluminal image associated with the location corresponding to the minimum stent area. In some aspects, the processor circuit is further configured to display a distance measurement corresponding to the distance between the stent edge and the vessel wall at the location corresponding to the minimum stent area. In some aspects, the processor circuit is further configured to display an intraluminal image of the plurality of intraluminal images. In some aspects, the processor circuit is further configured to display a marker along the longitudinal view corresponding to the location along the longitudinal view at which the intraluminal image was obtained. In some aspects, the processor circuit is further configured to display a distance measurement corresponding to the distance between the stent edge and the vessel wall associated with the intraluminal image. In some aspects, the distance between the stent edge and the vessel wall for the plurality of intraluminal images comprises an average distance between the stent edge and the vessel wall. In some aspects, the distance between the stent edge and the vessel wall for the plurality of intraluminal images comprises a maximum distance between the stent edge and the vessel wall. In some aspects, the processor circuit is configured to generate the longitudinal view of the body lumen based on the plurality of intraluminal images. In some aspects, the processor circuit is configured to generate the longitudinal view of the body lumen based on one or more measurements of the stent edge and the vessel wall within the plurality of intraluminal images.

In an exemplary aspect of the present disclosure, a method is provided. The method includes receiving, with a processor circuit in communication with an intraluminal imaging device, a plurality of intraluminal images obtained by the intraluminal imaging device during the movement of the intraluminal imaging device within a body lumen of a patient; identifying, with the processor circuit, a stent edge and a vessel wall within the plurality of intraluminal images; calculating, with the processor circuit, a distance between the stent edge and the vessel wall for the plurality of intraluminal images; comparing, with the processor circuit, the distance between the stent edge and the vessel wall with a threshold distance, for the plurality of intraluminal images; identifying, with the processor circuit, a subset of the plurality of intraluminal images corresponding to the distance between the stent edge and the vessel wall satisfying the threshold distance; outputting, to a display in communication with the processor circuit, a screen display comprising a longitudinal view of the body lumen, wherein the longitudinal view of the body lumen comprises: a graphical representation of the stent; and locations indicated along the stent corresponding to the subset of intraluminal images.

In an exemplary aspect of the present disclosure, a system is provided. The system includes an intravascular imaging device; and a processor circuit configured for communication with the intravascular imaging device, the processor circuit configured to: receive a plurality of intravascular images obtained by the intravascular imaging device during the movement of the intravascular imaging device within a blood vessel of a patient; identify a stent edge of a stent and a vessel wall within the plurality of intravascular images; calculate a distance between the stent edge and the vessel wall for the plurality of intravascular images; compare, for the plurality of intravascular images, the distance between the stent edge and the vessel wall with a threshold distance; identify a subset of the plurality of intravascular images corresponding to the distance between the stent edge and the vessel wall satisfying the threshold distance; output, to a display in communication with the processor circuit, a screen display comprising a longitudinal view of the blood vessel, wherein the longitudinal view of the blood vessel comprises: a graphical representation of the stent; and locations indicated along the stent corresponding to the subset of intravascular images.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 is a schematic diagram of a processor circuit, according to aspects of the present disclosure.

FIG. 3 is a diagrammatic view of a graphical user interface, according to aspects of the present disclosure.

FIG. 4 is a diagrammatic view of a longitudinal view of a vessel with a stent, according to aspects of the present disclosure.

FIG. 5 is a diagrammatic view of a graphical user interface, according to aspects of the present disclosure.

FIG. 6 is a diagrammatic view of a longitudinal view of a vessel with a stent, according to aspects of the present disclosure.

FIG. 7 is a flow diagram of a method of assessing stent deployment and automatically identifying regions of a stent corresponding to increased risk of post-stent complications, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

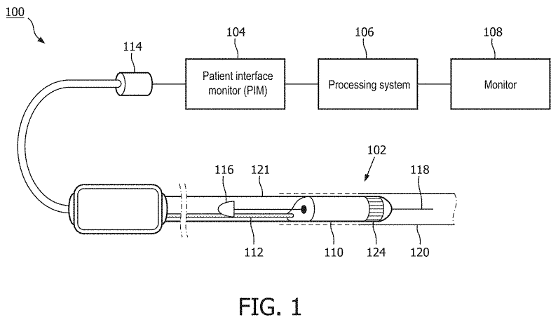

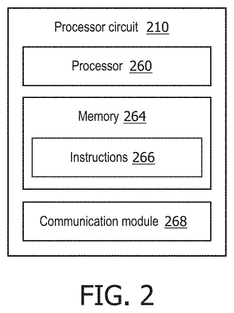

FIG. 1 is a diagrammatic schematic view of an intraluminal imaging system 100, according to aspects of the present disclosure. The system 100 may be a system comprising a processor circuit configured for communication with an intravascular imaging catheter 102 and a display 108. The system 100 may be a system comprising a processor circuit configured for communication with an intravascular ultrasound (IVUS) imaging catheter 102 and a display 108. The processor circuit may be the processor circuit 210 described with reference to FIG. 2. The display 108 may also be referred to as a monitor, as shown in FIG. 1. The intraluminal imaging system 100 can be an ultrasound imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, a processing system or console 106, and a monitor 108. The intraluminal imaging device 102 can be an ultrasound imaging device. In some instances, the device 102 can be an IVUS imaging device, such as a solid-state IVUS device. The intraluminal imaging device 102 may also be referred to as an intraluminal imaging catheter. The intraluminal imaging device may also be referred to as an intravascular ultrasound (IVUS) imaging catheter.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

The PIM 104 facilitates communication of signals between the IVUS console 106 and the scanner assembly 110 included in the IVUS device 102. This communication includes the steps of: (1) providing commands to integrated circuit controller chip(s) 206A and 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A and 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) 126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the transmission line bundle or cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

It is understood that the system 100 and/or device 102 can be configured to obtain any suitable intraluminal imaging data. In some embodiments, the device 102 may include an imaging component of any suitable imaging modality, such as optical coherence tomography (OCT), intracardiac echocardiography (ICE), etc.

FIG. 2 is a schematic diagram of a processor circuit, according to aspects of the present disclosure. The processor circuit 210 may be implemented in the processing system 106 of FIG. 1. In an example, the processor circuit 210 may be in communication with the intraluminal imaging device 102, the x-ray imaging system 109, and/or the display 108 within the system 100. The processor circuit 210 may include a processor and/or communication interface. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the probe 110 and/or the processing system 106 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the probe 110, and/or the display or monitor 108. In that regard, the communication module 268 can be an input/output (I/O) device. In some instances, the communication module 268 facilitates direct or indirect communication between various elements of the processor circuit 210 and/or the probe 110 (FIG. 1) and/or the processing system 106 (FIG. 1).

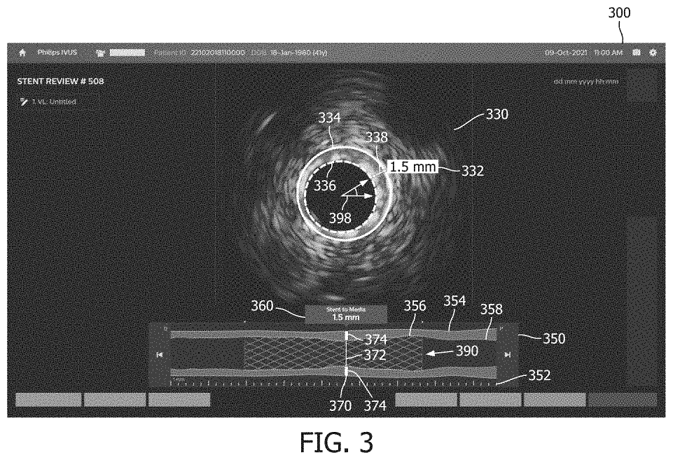

FIG. 3 illustrates a graphical user interface 300 according to aspects of the present disclosure. The interface 300 may include an IVUS image 330 and a longitudinal view 350.

In some embodiments, the processor circuit 210 (FIG. 2) may be configured to acquire intravascular ultrasound (IVUS) data during an IVUS pullback imaging procedure, as described with reference to FIG. 1. The processor circuit 210 may generate multiple IVUS images, like the image 330 shown, based on this received IVUS data. Each IVUS image received may include a radial cross-sectional view of a vessel of the patient. In some cases, a medical treatment device, such as a stent, may be present within the vessel imaged by the intravascular imaging system. Some of the received IVUS images may correspond to regions of the vessel which include a stent. As a result, a view of a stent edge may be visible within some of the received IVUS images, such as the IVUS image 330. The processor circuit 210 may be configured to analyze each generated IVUS image 330. The processor circuit 210 may automatically identify within each IVUS image a vessel wall as well as a stent edge if a depiction of a stent is determined to be present within the IVUS image.

In the IVUS image 330 shown in FIG. 3, a vessel wall 334 is shown. The processor circuit 210 may be configured to highlight or identify the vessel wall 334 in any suitable way. For example, the vessel wall 334 may be identified by a line of various colors, patterns, or other visual appearances or by another way. The circuit 210 may also generate one or more visual indicators which may identify the vessel wall 334. In some embodiments, the cross-sectional area defined by the vessel wall 334 may also be identified. For example, this region may be shaded with a color or pattern or other overlay.

In addition, in the IVUS image 330 shown in FIG. 3, a stent edge 336 is shown. The IVUS image 330 shown in FIG. 3 may be an IVUS image obtained by the intravascular imaging system at a region of the vessel corresponding to a stent. The processor circuit 210 may be configured to highlight or identify the stent edge 336 in any suitable way. For example, the stent edge 336 may be identified by a line of various colors, patterns, or other visual appearances. The circuit 210 may also generate one or more visual indicators which may identify the stent edge 336. In some embodiments, the cross-sectional area defined by the stent edge 336 may also be highlighted. For example, this region may be shaded with a color or pattern or other overlay. In some embodiments, the processor circuit 210 may identify the stent edge 336 in a different way from the vessel wall 334. In this way, a user of the system may distinguish the vessel wall 334 from the stent edge 336 identified in the IVUS image 330.

The processor circuit 210 may automatically identify the vessel wall 334 and/or the stent edge 336 of an IVUS image, such as the image 330. Examples of border detection (e.g., detection of a vessel wall and/or a stent edge), image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

The IVUS image 330 may also include an indicator 338 specifying a distance between the stent edge 336 and the vessel wall 334. The distance associated with the indicator 338 can be a radial distance. In some embodiments, the indicator 338 may be overlaid over the IVUS image 330 and may extend from one location along the stent edge 336 in an outward radial direction to the vessel wall 334. A distance between the stent edge 336 and the vessel wall 334 may be determined at any location within the IVUS image 330. Similarly, the indicator 338 may be placed at any location within the IVUS image and may illustrate any distance measurement between the stent edge 336 and the vessel wall 334 based on its location.

In some embodiments, a metric 332 may also be displayed. The metric 332 may be associated with the distance measurement shown by the indicator 338. For example, the metric 332 may be positioned within the graphical user interface 300 proximate to the indicator 338. In some embodiments, the metric 332 may alternatively be associated with an average distance between the stent edge 336 and the vessel wall 334 at multiple locations within the image 330. For example, a distance measurement between the stent edge 336 and the vessel wall 334 may be determined in any direction from the center of the IVUS image 330. For example, in one embodiment, the distance measurement 332 and indicator 338 may correspond to a distance measurement made at an angle 398 from a center point of the IVUS 330 or a location of the imaging device or other reference point. The angle 398 may be increased or decreased sequentially around a full circle and a similar distance measurement between the stent edge 336 and the vessel wall 334 may be made at each angle. Each of these distance measurements may be averaged. In some embodiments, the metric 332 may reflect this average distance. In other embodiments, the metric 332 may alternatively reflect a maximum distance measurement, a minimum distance measurement, a median, or any other suitable metric, value, or measurement. In some embodiments, other metrics or measurements may be displayed within the graphical user interface 300.

The graphical user interface 300 additionally may include a longitudinal intraluminal image 350. The longitudinal image 350 can be referred to as in-line digital (ILD) display or image longitudinal display (ILD). The IVUS images acquired during an intravascular ultrasound imaging procedure, such as during an IVUS pullback, may be used to create the ILD 350. In that regard, an IVUS image is a tomographic or radial cross-sectional view of the blood vessel. The ILD 350 provides a longitudinal cross-sectional view of the blood vessel. The ILD 350 can be a stack of the IVUS images acquired at various positions along the vessel, such that the longitudinal view of the ILD 350 is perpendicular to the radial cross-sectional view of the IVUS images. In such an embodiment, the ILD 350 may show the length of the vessel, whereas an individual IVUS image is a single radial cross-sectional image at a given location along the length. In another embodiment, the ILD 350 may be a stack of the IVUS images acquired overtime during the imaging procedure and the length of the ILD 350 may represent time or duration of the imaging procedure. The ILD 350 may be generated and displayed in real time or near real time during the pullback procedure. As each additional IVUS image is acquired, it may be added to the ILD 350. For example, at a point in time during the pullback procedure, the ILD 350 shown in FIG. 3 may be partially complete. In some embodiments, the processor circuit may generate an illustration of a longitudinal view of the vessel being imaged based on the received IVUS images. For example, rather than displaying actual vessel image data as the ILD 350 does, the illustration may be a stylized version of the vessel, with e.g., continuous lines showing the lumen border, a stent border, and/or the vessel border.

The ILD 350 may include a depiction of a vessel wall 354, a stent edge 356, a lumen border 358, and a stent 390. As an example, the ILD 350 shown in FIG. 3 may be a stylized ILD. Specifically, the depiction of the vessel wall 354, the lumen border 358, and the stent 356 may be positioned along the ILD 350 based on diameter measurements in corresponding IVUS images. For example, at each location along the length of the imaged vessel, an average diameter of the lumen border, the stent edge, and the vessel wall may be calculated. Calculating an average diameter may include any of the features described with reference to calculating an average distance measurement described with reference to the metric 332. Specifically, a distance measurement in all directions from the center of the IVUS image 330 may be made. These measurements may then be averaged. As shown in the ILD 350, continuous lines corresponding to the vessel wall 354, the lumen border 358, and the stent edge 356 may be positioned symmetrically around a center, longitudinally extending line with a distance between the upper and lower lines of the lumen borders 358, stent edge 356, and vessel wall 354 reflecting the averaged diameter for each respective metric.

As shown in FIG. 3, the ILD 350 may additionally include a scale 352. The scale 352 may illustrate relative distances longitudinally along the ILD 350 such that size and distance measurements of various aspects of the ILD 350 may be more easily compared and/or visually quantified.

In some embodiments, aspects of the present disclosure may include a coregistration process. For example, a coregistration process may include performing an IVUS imaging pullback procedure while also acquiring extraluminal images, such as no-contrast x-ray images, including fluoroscopy images and/or cine images. In particular, during an IVUS imaging procedure, a physician may position an IVUS imaging device (e.g., the device 102 of FIG. 1) at a distal position within a vessel. After or before the IVUS imaging device is positioned at this distal location, the physician may direct an extraluminal imaging system, such as an x-ray system, to begin continuously obtaining extraluminal images of the patient anatomy such that the extraluminal images show a radiopaque portion of the IVUS imaging device in the vessel. The physician may then direct the IVUS imaging device to begin to acquire IVUS images. In that regard, both the IVUS imaging device and the extraluminal imaging device may receive respective images simultaneously. The IVUS imaging device may be moved through the vessel. As the imaging device moves, it may acquire IVUS images while the extraluminal device acquires extraluminal images.

After the pullback is complete, the system may identify the location of one or more radiopaque markers of the IVUS imaging device within each acquired extraluminal image. In this way, each extraluminal image may be associated with at least one position of the IVUS device. These positions, which may be stored as coordinates of pixels within the extraluminal image, may define a pathway of the IVUS imaging device. Each location along this pathway may be associated with one or more IVUS images obtained by the IVUS imaging device at that location along the pathway as seen in an extraluminal image. The IVUS imaging device pathway may then be overlaid over an extraluminal image, such as one of the extraluminal images obtained previously, or a separate extraluminal image (e.g., a contrast-filled angiogram image). As a result, in some embodiments, a user of the system may advantageously be able to see a location within an extraluminal image of the vessel associated with an IVUS image obtained at that location. Based on this same location data, the scale 352 shown associated with the ILD 350 may be based on locations (e.g., in mm) of IVUS images received.

In some embodiments, the scale 352 shown along the ILD 350 may be determined based on the coregistered positions of IVUS images on an extraluminal image. For example, distances between the locations of IVUS images may be determined by the processor circuit 210 within an extraluminal image. In some embodiments, radiopaque markers of the IVUS imaging device may be of a known distance. This distance may provide a reference distance. For example, a number of pixels within the extraluminal image corresponding to the distance between radiopaque markers may be determined and used to determine any other distance measurements within the extraluminal image. As an example, distances between any obtained IVUS images may be known and displayed visually by the scale 352.

The ILD 350 may also include a depiction of the stent 390. This depiction may include various visual characteristics of the stent to distinguish the border or lumen of the stent 390 from other aspects of the ILD 350. As an example, one particular pattern may be overlaid over the lumen area of the stent 390. A line 356 may identify the stent edge. The ILD 350 may additionally include an indicator 370. The indicator 370 may represent the location at which the IVUS image 330 displayed was acquired. The indicator 370 may include one or more elements 374 and an element 372. The element 372 may identify a diameter of the lumen at the location of the indicator 370. The indicators 374 may identify a region between the stent edge 356 and the vessel wall 354. The indicators 374 may include elements on either side of the lumen shown in the ILD 350. In some embodiments, the indicators 374 may be visually differentiated from the indicator 372. For example, the indicator 374 may include various visual characteristics such as varying colors, patterns, shapes, widths, or other visual depictions which differentiate the indicators 374 from the indicator 372.

The ILD 350 may additionally include an element 360. The element 360 may display to user a distance measurement from the stent edge 356 to the vessel wall 354. The element 360 may correspond to a distance measurement shown by the indicators 374. This distance measurement 360 may be the same measurement as the metric 332 discussed previously. The element 360 may include any other suitable label, title, or any suitable alphanumeric text. In some embodiments, the distance from the stent edge 356 to the vessel wall 354 as shown in the ILD 350, or the distance from the stent edge 336 to the vessel wall 334 of the IVUS image 330, may be a distance from the stent edge to the media-adventitia of the vessel wall. Alternatively, the vessel wall may be defined by the intima, media, adventitia, external elastic lamina, or any other structure of a blood vessel.

In some embodiments, the processor circuit 210 may be configured to automatically identify locations along an imaged vessel at which the distance between a stent edge and the vessel wall exceeds a threshold amount. This automatic identification may advantageously assist a user of the system, or a physician, to determine the likelihood of complications related to the stent in the future. For example, the distance between the stent edge and the vessel wall may affect the likelihood that a stent will become dislodged or malapposed in the future. In some embodiments, a threshold distance between a stent edge and a vessel wall may be determined. For example, the processor circuit 210 may determine a threshold value according to past data relating to the same patient or from different patients. The processor circuit 210 may receive a threshold value based on recommendations from experts in the field, or may receive a threshold value as an input from the user. In some embodiments, this threshold value may be 0.5 millimeters. In some embodiments, this threshold may be more or less than this value. In some embodiments, the threshold value may be adjusted by a user of the system.

The processor circuit 210 (FIG. 2) may be configured to compare a distance measurement, such as the measurement shown by the indicator 338, to the received threshold distance value. In some embodiments, the processor circuit 210 may compare any distance measurement with the threshold. For example, the processor circuit 210 may be configured to compare an average distance between the stent and the vessel wall with the threshold. This average distance may reflect all distances calculated between the stent edge and the vessel wall at various locations within the image 330. In other embodiments, the processor circuit 210 may be configured to compare a maximum distance associated with an IVUS image with the threshold. In some embodiments, the processor circuit may compare a minimum distance with the threshold. Any other suitable distance measurement between a stent edge and a vessel wall may also be compared to the threshold value.

As discussed previously, a distance between a stent edge and a vessel wall may be indicative of complications after a stent is positioned. In addition, the processor circuit 210 may be configured to determine a plaque burden associated with each received IVUS image. The plaque burden associated with each IVUS image may serve as an additional factor in determining the likelihood of complications of a stent. For example, after the processor circuit 210 has determined the locations of a stent edge 336 and a vessel wall 334, the processor circuit 210 may be configured to determine a plaque burden. In some embodiments, the processor circuit 210 may be configured to identify a lumen boundary within an IVUS image, such as the image 330 shown in FIG. 3. Based on the location of the lumen boundary in each IVUS image, the processor circuit 210 may calculate a cross sectional area of the lumen within each received IVUS image. Similarly, based on the location of the vessel wall in each IVUS image, the processor circuit 210 may additionally calculate a cross sectional area of the vessel within each received IVUS image. The processor circuit 210 may then use these two calculations of cross sectional area to determine a plaque burden associated with each IVUS image. In one example, the processor circuit 210 may divide the cross sectional area of the lumen by the cross sectional area of the vessel. This value may be converted to a percentage or a ratio and stored in a memory in communication with the processor circuit 210 as a plaque burden. The processor circuit 210 may additionally be configured to identify locations along the imaged vessel, or locations along the stent, that may be associated with a greater risk of future complications based on an increased plaque burden when compared with other locations along the stent or a threshold plaque burden. The processor circuit 210 may determine a threshold plaque burden value in any suitable way. For example, the processor circuit 210 may determine a threshold value according to past data relating to the same patient or to different patients, may receive a threshold value based on recommendations from experts in the field, or may receive a threshold value as an input from the user.

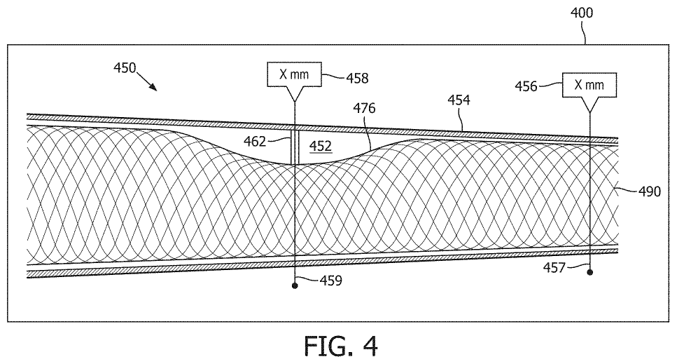

FIG. 4 is a diagrammatic view of a longitudinal view 450 of a vessel with a stent 490, according to aspects of the present disclosure. The longitudinal view 450 shown in FIG. 4 may be an ILD 450. The ILD 450 may include an indicator 459 and an indicator 457.

The indicator 459 may be substantially similar to the indicator 370 of FIG. 3. For example, the indicator 459 may identify to a user of the system the location at which an IVUS image was acquired. The indicator 459 may similarly correspond to a metric 458. The metric 458 may represent a distance between a stent edge 476 to a vessel wall 454. For example, as shown in FIG. 4, the ILD 450 may include a depiction of a stent 490. The stent 490 may be positioned within a vessel. The vessel may include a vessel wall 454. The vessel wall 454 may be a vessel media-adventitia. The ILD 450 may additionally depict tissue 452. The tissue 452 may include various materials within the vessel. In some examples, the tissue 452 may include plaque, such as calcified deposits (e.g., calcium), dense calcium, fibrous tissues, fibro-fatty tissues, lipids, complex carbohydrates, necrotic core, various blood cells, dead blood cells, muscle cells, or other materials. The indicator 459 may be displayed to indicate for a user a location along the ILD 450 at which an increase or a maximum in the radial cross-sectional area of tissue 452 is present. In some embodiments, an increase or maximum in the radial cross-sectional area of the tissue 452 may correspond to a decrease or minimum in the radial cross-section area of the lumen of the vessel (e.g., the luminal area). For example, the indicator 459 may correspond to a location along the ILD 450 at which a distance between the stent edge and the vessel wall exceeds a threshold or is at a maximum.

As shown in FIG. 4, a location corresponding to a distance between the stent edge and the vessel wall exceeding a threshold may or may not be the same as the location along the stent 490 as the minimum stent area. For example, the indicators 457 and 456 (or any other indicators) may identify a location along the ILD 450 of a minimum stent area (MSA).

In some embodiments, as previously described with reference to FIG. 3, a plaque burden may be calculated for any received IVUS images. In some embodiments, the processor circuit 210 (FIG. 2) may be configured to calculate a plaque burden at the location of minimum stent area. In the example shown in FIG. 4, a plaque burden of the IVUS image acquired at the location of the indicator 457 and/or the indicator 456 may be determined. In some cases, if the plaque burden for this location is determined to be above a threshold plaque burden, it may correspond to an increased risk of the stent coming dislodge or not being placed well. Either of these warnings, as well as any other applicable conditions or predicted conditions, may be displayed or otherwise conveyed to a user by the processor circuit 210 (FIG. 2).

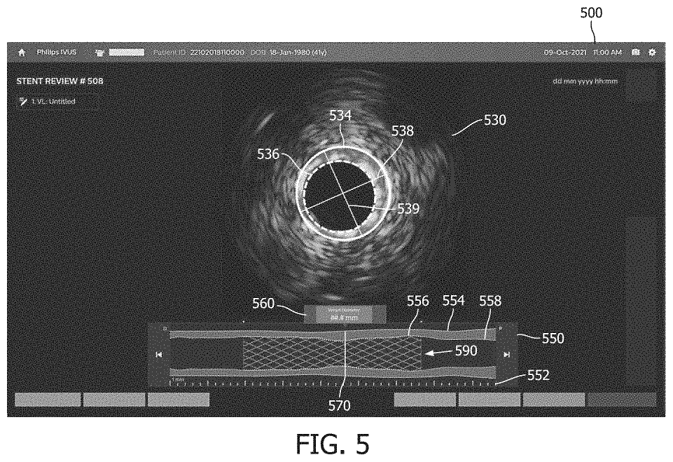

In the example shown in FIG. 4, the ILD 450 may be an image-based ILD. In other embodiments, the ILD may be a stylized ILD, such as in the examples shown in FIG. 3 and FIG. 5. A stylized ILD may be a measurement-based ILD. For example, a measurement-based ILD may include a depiction of various boundaries or structures of a vessel in a longitudinal view. The relative positions, sizes, and/or other appearances of the parts of the measurement-based ILD may be determined based on one or more measurements of the vessel. These measurements may include distance measurements in a longitudinal direction with the vessel, distance measurements between various boundaries, such as a distance from lumen boundaries of the vessel, a stent edge to a vessel wall, a lumen boundary to a vessel wall, or any other distance measurements. The measurements may also include any other measurements obtained based on an intraluminal or extraluminal image of the vessel or other physiological measurements.

In some embodiments, the distance between a stent and a vessel wall should be minimalized. For example, the distance between the stent edge 476 and the vessel wall 454, as shown by the indicator 462, should be minimalized. In some embodiments, a larger distance between the stent edge 476 and the vessel wall 454 may correspond to an increased likelihood of future complications of the stent. In some embodiments, a distance between the stent edge 476 and the vessel wall 454 being larger than a threshold may additionally or alternatively indicate that a post-dilatation procedure is recommended.

FIG. 5 is a graphical user interface 500, according to aspects of the present disclosure. The graphical user interface 500 includes an IVUS image 530 and an ILD 550. The graphical user interface 500 shown in FIG. 5 illustrates various methods of calculating a vessel diameter, a cross-sectional diameter of a stent, and/or a lumen diameter for each IVUS image.

The IVUS image 530 shown in FIG. 5 may include a depiction of a vessel wall 534, and a stent edge 536. A diameter 538 and a diameter 539 are also shown within the IVUS image 530. In any IVUS image, the diameter of a vessel may vary depending on the direction or location at which the diameter measurement is made. In other words, the cross-sectional shape of a vessel shown in an IVUS image may not be circular. As a result, a distance from one location of the vessel wall across the lumen to the other side of the vessel wall may vary. For example, the diameter measurement 538 shown in FIG. 5 may differ from the diameter measurement 539. This may be because the cross-sectional area of a vessel may or may not be perfectly circular. To account for irregular cross-sectional shapes of various images, an average diameter may be calculated. As an example, multiple diameter measurements, such as the diameter of 538 and/or the diameter measurement 539, as well as other measurements, may be made within an IVUS image. In some embodiments, all diameter measurements may be averaged to determine an average diameter measurement associated with a vessel wall within an IVUS image.

In one example, the processor circuit 210 may be configured to determine an average diameter measurement of each IVUS image received during an imaging procedure. The processor circuit 210 may additionally be configured to identify a location along the imaged vessel corresponding to a minimum stent area. For example, as has been previously described, the processor circuit 210 may be configured to identify a stent edge within each received IVUS image. Based on the identification of a stent edge within an image, the processor circuit 210 may also determine a cross sectional area of the lumen, or in some embodiments, a cross sectional area of the stent within the image. A stent cross sectional area may thus be associated with each received IVUS image and/or each location along the image vessel. The processor circuit 210 may then identify a location along the imaged vessel and a corresponding IVUS image associated with a minimum stent area.

In some embodiments, a physician may desire to know the average vessel diameter at the location of the minimum stent area. This value may influence the decision to deploy various treatment devices or procedures to expand the stent area at that location. For example, an angioplasty balloon may be deployed at the location of minimum stent area to reexpand a stent (post-dilatation) and further restore blood flow. The average vessel diameter at the location of the minimum stent area may assist a physician in determining whether to deploy such a device, as well as to determine to what extent the stent may be reexpanded.

As shown in FIG. 5, the ILD 550 may include various features or elements. For example, the ILD 550 may include a depiction of a vessel wall 554, a depiction of the lumen border 558, a depiction of the stent edge 556, and a depiction of a stent 590. The ILD 550 may additionally include a scale 552. The scale 552 may be substantially similar to the scale 352 described with reference to FIG. 3. The ILD 550 may additionally include an indicator 570. The indicator 570 may display for a user the location along the ILD 550 at which the IVUS image 530 was obtained. In the example shown in FIG. 5, the indicator 570 may also identify a location of the imaged vessel corresponding to a minimum stent area.

An element 560 may also be displayed proximate to the indicator 570 of the ILD 550. The element 560 may display for a user the vessel diameter at the location identified by the indicator 570. The element 560 may be displayed at any location within the graphical user interface 500. The element 560 may also be of any suitable visual appearance. The vessel diameter measurement may refer to an average vessel diameter, a maximum vessel diameter, a minimum diameter, a cross-sectional area, or any other measurement associated with the vessel wall 534 and/or the stent wall 536 identified within the image 530. In some embodiments, other diameters, cross-sectional areas, or any other measurements may be made by the processor circuit 210. For example, the element 560 may refer to a lumen diameter, including an average lumen diameter, a maximum lumen diameter, or a minimum lumen diameter, or a cross-sectional area of the lumen. The element 560 may refer to a stent diameter, including an average stent diameter, a maximum stent diameter, or a minimum stent diameter, or a cross-sectional area of the stent.

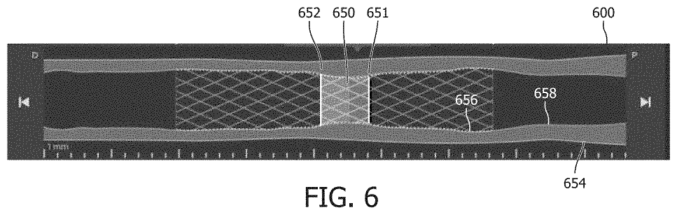

FIG. 6 depicts an ILD 600. The ILD 600 may be displayed to a user separately from or simultaneously with any of the longitudinal views described herein. For example, the ILD 600 may be displayed as an element of the graphical user interface 300 or the graphical user interface 500.

The ILD 600 includes a depiction of the vessel wall 654, the lumen border 658, and the stent edge 656. The ILD 600 additionally shows a region 650. The region 650 may identify all locations along the vessel representative of an increased risk of future stent complications. For example, in one embodiment, the region 650 may identify locations along the vessel corresponding to a distance or an average distance between the stent edge 656 and the vessel wall 654 exceeding a threshold, such as 0.5 mm. In another embodiment, the region 650 may identify locations along the vessel corresponding to a plaque burden exceeding a threshold plaque burden.

The region 650 may be differentiated from other regions of the ILD 600 in any suitable way. For example, the region 650 may include a proximal indicator 651 and a distal indicator 652 identifying distal and proximal ends of the region 650. The region 650 may also include a region of varied color, shading, pattern, transparency, or other visual differentiation to distinguish the region 650 from other parts of the ILD 600. In some embodiments, the region 600 may correspond to a single continuous length of the ILD 600. In other embodiments, the region 600 may include multiple regions separated by areas of the vessel at which either the distance between the stent edge and the vessel wall or the plaque burden do not exceed thresholds.

In some embodiments in which the intravascular imaging data has been co-registered to an x-ray image, the region 650 can also be overlaid on the corresponding region of the vessel in the x-ray image. For example, in a corresponding extraluminal image, a view of the same vessel and/or stent may be displayed. A section of the vessel corresponding to the same location of the region 650 in the ILD 600 may be highlighted or otherwise differentiated within the extraluminal image.

Regions along the stent or vessel corresponding to a need of post-dilatation or an increased risk of future complications may be conveyed to a user of the system in a number of ways. In one example, regions in need of a post-dilatation procedure may be identified by regions along a longitudinal view of a vessel (e.g., the ILD 600). For example, various indicators (e.g., the region 650 and/or the indicators 651 and/or 652 may be used to identify regions in need of post-dilatation procedures. The locations of these regions may be determined by any of the methods described in the present disclosure.

In another example, regions along a stent corresponding to an increased risk of post-deployment complications may be identified by regions along a longitudinal view of a vessel (e.g., the ILD 600). Any of the same indicators may identify for a user these regions. The locations of these regions may be determined by any of the methods described in the present disclosure.

In another example, an indicator may be displayed which conveys to the user that an entire stent is in need of a post-dilatation procedure or is at risk of further complications. For example, an alpha-numeric indicator including any suitable text or visual depictions including images or other notification including a visual, audio, or haptic notification may be output. Any of these indicators may correspond to a stent. Any of these indicators may convey to the user that the stent as a whole is either in need of a post-dilatation procedure or is at an increased risk of further complications.

In another example, the processor circuit may be configured to calculate a likelihood of future complications. In some embodiments, the processor circuit may determine a likelihood of future complications of a stent based on a distance measurement between the stent and the vessel wall, a diameter of the vessel (e.g., the vessel lumen or any border of the vessel wall), a diameter of the stent, cross-sectional area of the vessel, cross-sectional area of the stent, and/or a plaque burden value of an IVUS image and/or a location along the vessel or stent. For example, the processor circuit can perform an in-stent restenosis prediction, as described in U.S. Pat. No. 10,772,599, titled “Devices, systems, and methods for in-stent restenosis prediction”, which is incorporated by reference herein in its entirety.

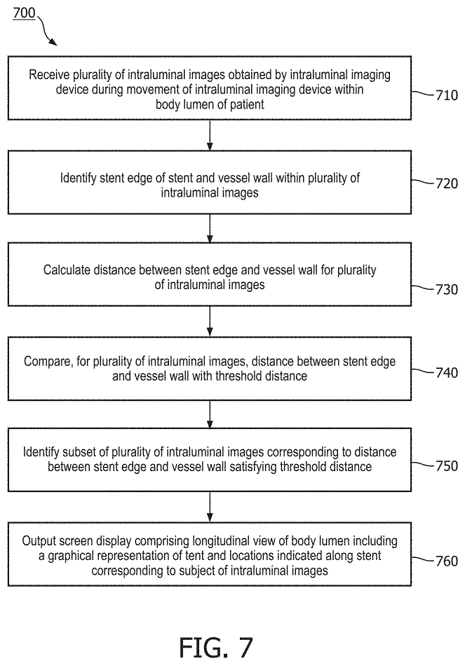

FIG. 7 is a flow diagram of a method 700 of assessing stent deployment and automatically identifying regions of a stent corresponding to increased risk of post-stent complications, according to aspects of the present disclosure. The method 700 may describe an automatic segmentation of a vessel to detect segments of interest using co-registration of invasive physiology and x-ray images. As illustrated, the method 700 includes a number of enumerated steps, but embodiments of the method 700 may include additional steps before, after, or in between the enumerated steps. In some embodiments, one or more of the enumerated steps may be omitted, performed in a different order, or performed concurrently. The steps of the method 700 can be carried out by any suitable component within the diagnostic system 100 and all steps need not be carried out by the same component. In some embodiments, one or more steps of the method 700 can be performed by, or at the direction of, a processor circuit of the diagnostic system 100, including, e.g., the processor 260 (FIG. 2) or any other component.

At step 710, the method 700 includes receiving a plurality of intraluminal images obtained by the intraluminal imaging device during the movement of the intraluminal imaging device within a body lumen of a patient. In some aspects, step 710 may include receiving a plurality of IVUS images obtained by the IVUS imaging device during the movement of the IVUS imaging device within a blood vessel of a patient.

At step 720, the method 700 includes identifying a stent edge of a stent and a vessel wall within the plurality of intraluminal images. In some aspects, step 720 may include identifying a stent edge of a stent and a vessel wall within the plurality of IVUS images.

At step 730, the method 700 includes calculating a distance between the stent edge and the vessel wall for the plurality of intraluminal images. In some aspects, step 730 may include calculating a distance between the stent edge and the vessel wall for the plurality of IVUS images.

At step 740, the method 700 includes comparing, for the plurality of intraluminal images, the distance between the stent edge and the vessel wall with a threshold distance. In some aspects, step 740 may include comparing, for the plurality of IVUS images, the distance between the stent edge and the vessel wall with a threshold distance

At step 750, the method 700 includes identifying a subset of the plurality of intraluminal images corresponding to the distance between the stent edge and the vessel wall satisfying the threshold distance. In some aspects, step 750 may include identifying a subset of the plurality of IVUS images corresponding to the distance between the stent edge and the vessel wall satisfying the threshold distance. Satisfying the threshold distance may include exceeding a threshold distance, meeting or being equal to a threshold distance, or, in some cases, being less than a threshold distance.

At step 760, the method 700 includes outputting, to a display in communication with the processor circuit, a screen display comprising a longitudinal view of the body lumen. The longitudinal view of the body lumen includes a graphical representation of the stent and locations indicated along the stent corresponding to the subset of intraluminal images. In some aspects, step 760 may include outputting, to a display in communication with the processor circuit, a screen display comprising a longitudinal view of the blood vessel. The longitudinal view of the blood vessel includes a graphical representation of the stent; and locations indicated along the stent corresponding to the subset of IVUS images.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an intraluminal imaging device, the processor circuit configured to:
receive a plurality of intraluminal images obtained by the intraluminal imaging device during the movement of the intraluminal imaging device within a body lumen of a patient;
identify a stent edge of a stent and a vessel wall within the plurality of intraluminal images;
calculate a distance between the stent edge and the vessel wall for the plurality of intraluminal images;
compare, for the plurality of intraluminal images, the distance between the stent edge and the vessel wall with a threshold distance;
identify a subset of the plurality of intraluminal images corresponding to the distance between the stent edge and the vessel wall satisfying the threshold distance;
output, to a display in communication with the processor circuit, a screen display comprising a longitudinal view of the body lumen, wherein the longitudinal view of the body lumen comprises:
a graphical representation of the stent; and
locations indicated along the stent corresponding to the subset of intraluminal images.

2. The system of claim 1, wherein the processor circuit is further configured to automatically analyze the plurality of intraluminal images to determine whether the stent edge is present within the plurality of intraluminal images.

3. The system of claim 2, wherein the processor circuit is configured to perform the identification of the stent edge and the vessel wall and the calculation of the distance between the stent edge and the vessel wall in response to automatically determining that the stent edge is present in the plurality of intraluminal images.

4. The system of claim 1, wherein the processor circuit is further configured to determine a stent area for the plurality of intraluminal images.

5. The system of claim 4, wherein the processor circuit is further configured to identify a location along the longitudinal view corresponding to a minimum stent area.

6. The system of claim 5, wherein the processor circuit is further configured to display an intraluminal image associated with the location corresponding to the minimum stent area.

7. The system of claim 5, wherein the processor circuit is further configured to display a distance measurement corresponding to the distance between the stent edge and the vessel wall at the location corresponding to the minimum stent area.

8. The system of claim 1, wherein the processor circuit is further configured to display an intraluminal image of the plurality of intraluminal images.

9. The system of claim 8, wherein the processor circuit is further configured to display a marker along the longitudinal view corresponding to the location along the longitudinal view at which the intraluminal image was obtained.

10. The system of claim 6, wherein the processor circuit is further configured to display a distance measurement corresponding to the distance between the stent edge and the vessel wall associated with the intraluminal image.

11. The system of claim 1, wherein the distance between the stent edge and the vessel wall for the plurality of intraluminal images comprises an average distance between the stent edge and the vessel wall.

12. The system of claim 1, wherein the distance between the stent edge and the vessel wall for the plurality of intraluminal images comprises a maximum distance between the stent edge and the vessel wall.

13. The system of claim 1, wherein the processor circuit is configured to generate the longitudinal view of the body lumen based on the plurality of intraluminal images.

14. The system of claim 1, wherein the processor circuit is configured to generate the longitudinal view of the body lumen based on one or more measurements of the stent edge and the vessel wall within the plurality of intraluminal images.

15. A method, comprising:
receiving, with a processor circuit in communication with an intraluminal imaging device, a plurality of intraluminal images obtained by the intraluminal imaging device during the movement of the intraluminal imaging device within a body lumen of a patient;
identifying, with the processor circuit, a stent edge and a vessel wall within the plurality of intraluminal images;
calculating, with the processor circuit, a distance between the stent edge and the vessel wall for the plurality of intraluminal images;
comparing, with the processor circuit, the distance between the stent edge and the vessel wall with a threshold distance, for the plurality of intraluminal images;
identifying, with the processor circuit, a subset of the plurality of intraluminal images corresponding to the distance between the stent edge and the vessel wall satisfying the threshold distance;
outputting, to a display in communication with the processor circuit, a screen display comprising a longitudinal view of the body lumen, wherein the longitudinal view of the body lumen comprises:
a graphical representation of the stent; and
locations indicated along the stent corresponding to the subset of intraluminal images.

16. A system, comprising:
an intravascular imaging device; and
a processor circuit configured for communication with the intravascular imaging device, the processor circuit configured to:
receive a plurality of intravascular images obtained by the intravascular imaging device during the movement of the intravascular imaging device within a blood vessel of a patient;
identify a stent edge of a stent and a vessel wall within the plurality of intravascular images;
calculate a distance between the stent edge and the vessel wall for the plurality of intravascular images;
compare, for the plurality of intravascular images, the distance between the stent edge and the vessel wall with a threshold distance;
identify a subset of the plurality of intravascular images corresponding to the distance between the stent edge and the vessel wall satisfying the threshold distance;
output, to a display in communication with the processor circuit, a screen display comprising a longitudinal view of the blood vessel, wherein the longitudinal view of the blood vessel comprises:
a graphical representation of the stent; and
locations indicated along the stent corresponding to the subset of intravascular images.
